(19) United States
(12) Patent Application Publication
(10) Pub. No.: US 2022/0023282 A1
(43) Pub. Date: Jan. 27, 2022

(54) Compositions of Bedaquiline, Combinations Comprising Them, Processes for Their Preparation, Uses and Methods of Treatment Comprising Them

(71) Applicant: MannKind Corporation, Westlake Villiage, CA (US)

(72) Inventors: Thomas Hofmann, Doylestown, PA (US); Stefan Ufer, Raleigh, NC (US); Kevin Stapleton, Laguna Beach, CA (US)

(73) Assignee: MannKind Corporation, Westlake Villiage, CA (US)

(21) Appl. No.: 17/309,624

(22) PCT Filed: Dec. 9, 2019
(86) PCT No.: PCT/US2019/065144

§ 371 Date: Jun. 10, 2021

Related U.S. Application Data
(60) Provisional application No. 62/778,953, filed on Dec. 13, 2018.

(51) Int. Cl. A61K 31/47; A61K 9/00; A61J 7/00; A61K 45/06; A61K 31/498; A61P 31/04

(52) U.S. Cl. CPC A61K 31/47; A61K 9/0078; A61K 9/0075; A61P 31/04; A61K 45/06; A61K 31/498; A61J 7/0076

(57) ABSTRACT

The present invention relates to pharmaceutical compositions for inhalation comprising a therapeutically effective dose of bedaquiline wherein the bedaquiline is provided in the form of a suspension, or in which the bedaquiline is provided in the form of a dry powder, and processes for their preparation. Furthermore, the present invention provides pharmaceutical combinations comprising bedaquiline in the form of an aerosol for pulmonary inhalation. The combinations and compositions provided by the present invention may be used in the treatment and/or prophylaxis of pulmonary infections caused by mycobacteria and other gram-positive bacteria.

The present application is a national stage application of PCT/US2019/065144, filed Dec. 9, 2019, which claims the benefit of U.S. Provisional Application No. 62/778,953, filed Dec. 13, 2018, the content of which are incorporated herein by reference in their entirety.

### FIELD OF THE INVENTION

The present invention relates to pharmaceutical compositions for inhalation comprising a therapeutically effective dose of bedaquiline, wherein the bedaquiline is provided in the form of a suspension or as a dry powder; processes for their preparation; and uses and methods of treatment comprising them. Furthermore, the present invention provides pharmaceutical combinations comprising bedaquiline in the form of an aerosol for pulmonary inhalation.

The combinations and compositions provided by the present invention may be used in the treatment and/or prophylaxis of pulmonary infections caused by mycobacteria and other gram-positive bacteria.

### BACKGROUND OF THE INVENTION

Janssen Pharmaceutica (a subsidiary of J&J) discovered bedaquiline (initially referred to as TMC207) around 2002 while screening for compounds that would kill Mycobacterium smegmatis, a saprophytic distant relative of Mycobacterium tuberculosis. Bedaquiline (BDQ) emerged from a whole-cell screen of 70,000 library compounds against the nonpathogenic M. smegmatis strain of TB (see, for example Guillemont, J., Meyer, C., Poncelet, A., Bourdrez, X. and Andries, K., “Diarylquinolines, synthesis pathways and quantitative structure-activity relationship studies leading to the discovery of TMC207”, Future Medicinal Chemistry (2011), 3: pp. 1345-1360) where the racemic mixture (comprising four diastereomers) was shown to have useful activity against both M. smegmatis and M. tuberculosis, with the R, S enantiomer being the most potent. Bedaquiline (marketed under the brandname Sirturo™) falls into the class of compounds known as diarylquinolines (DARQs), also referred to as substituted quinoline derivatives.

Chemical names for bedaquiline include:
3-quinolineethanol, 6-bromo-a-[2-(dimethylamino)ethyl]-2-methoxy-a-1-naphthalenyl-β-phenyl-, (aS,βR)-; and
(1R,2S)-1-(6-bromo-2-methoxyquinolin-3-yl)-4-(dimethylamino)-2-(naphthalen-1-yl)-1-phenylbutan-2-ol.

The structure of bedaquiline (BDQ) is shown below.

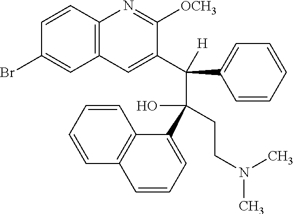 [SMILES: COc1nc2ccc(Br)cc2cc1[C@@H](c1ccccc1)[C@@](O)(CCN(C)C)c1cccc2ccccc12]

Structurally and mechanistically, DARQs are different from both fluoroquinolones (including methoxyquinolines) and other quinoline classes (see, for example, Andries, K., Verhasselt, P., Guillemont, J., Göhlmann, H W H., Neefs, J M., Winkler, H., Van Gestel, J., Timmerman, P., Zhu, M., Lee, E., Williams, P., de Chaffoy, D., Huitric, E., Hoffner, S., Cambau, E., Truffot-Pernot, C., Lounis, N. and Jarlier, V., “A diarylquinoline drug active on the ATP synthase of Mycobacterium tuberculosis”, Science (2005), 307: pp. 223-227). In vitro studies have shown that bedaquiline offers a new mechanism of anti-tuberculosis action by specifically inhibiting mycobacterial adenosine triphosphate (ATP) synthase.

Bedaquiline is also very lipophilic (measured log P 7.25), which may contribute to its induction of phospholipidosis, seen at high doses in preclinical models (see, for example, Mesens, N., Verbeeck, J., Rouan, M. and Vanparys, P., “Elucidating the role of M2 in the preclinical safety profile of TMC207. In Abstract on the 38th Union World Conference on Lung Health, Cape Town, South Africa, 2007). Its high lipophilicity may also contribute to bedaquiline's long terminal elimination half-life (see, for example, Svensson, E M., Murray, S., Karlsson, M O. and Dooley, K E., “Rifampicin and rifapentine significantly reduce concentrations of bedaquiline, a new anti-TB drug”, Journal of Antimicrobial Chemotherapy (2015), 70: pp. 1106-1114), which may lead to tissue overproportional accumulation at high doses or with daily dosing. More significantly, bedaquiline has been shown to potentially inhibit drug sensitive tuberculosis, multi-drug resistant tuberculosis and latent tuberculosis and is the first drug to be approved by the Food and Drug Administration for tuberculosis treatment in 40 years.

Impressive Phase llb clinical studies demonstrated that the addition of bedaquiline to tuberculosis treatment regimens significantly improved cure rates, reduced relapse rates, and reduced the duration of treatment compared to conventional regimens alone (see, for example, Diacon, A H., Pym, A., Grobusch, M., Patientia, R., Rustomjee, R., Page-Shipp, L., Pistorius, C., Krause, R., Bogoshi, M., Churchyard, G., Venter, A., Allen, J., Palomino, J C., De Marez, T., van Heeswijk, R P G., Lounis, N., Meyvisch, P., Verbeeck, J., Parys, W., de Beule, K., Andries, K. and Mc Neeley, D F., “The Diarylquinoline TMC207 for Multidrug-Resistant Tuberculosis”, The New England Journal of Medicine (2009), 360: pp. 2397-2405; Diacon, A H., Dawson, R., van Groote-Bidlingmaier, F., Symons, G., Venter, A., Donald, P R., van Niekerk, C., Everitt, D., Winter, H., Becker, P., Mendel, C M. and Spigelman, M K., “14-day bactericidal activity of PA-824, bedaquiline, pyrazinamide, and moxifloxacin combinations: a randomised trial”, The Lancet (2012), 380(9846): pp. 986-993; Pym, A S., Diacon, A H., Tang, S J., Conradie, F., Danilovits, M., Chuchottaworn, C., Vasilyeva, I., Andries, K., Bakare, N., De Marez, T., Haxaire-Theeuwes, M., Lounis, N., Meyvisch, P., Van Baelen, B., van Heeswijk, R P G. and Dannemann, B., “Bedaquiline in the treatment of multidrug- and extensively drugresistant tuberculosis”, The European Respiratory Journal (2016), 47(2): pp. 564-574). Importantly, BDQ retains clinical activity against drug-susceptible, multi-drug resistant, and extensively-drug resistant TB.

Bedaquilines's antimicrobial activity (Soni, I., De Groote, M A., Dasgupta, A. and Chopra, S., “Challenges facing the drug discovery pipeline for non-tuberculous mycobacteria”, Journal of Medical Microbiology (2016), 65: pp. 1-8) is unique among antibiotics due to its specificity, and potency, towards mycobacteria alone, by inhibiting mycobacterial ATP synthase (Koul, A., Dendouga, N., Vergauwen, K., Molenberghs, B., Vranckx, L., Willebrords, R., Ristic, Z., Lill, H., Dorange, I., Guillemont, J., Bald, D. and Andries, K., “Diarylquinolines target subunit c of mycobacterial ATP synthase”, Nature Chemical Biology (2007), 3: pp. 323-324). Indeed, Andries et al. demonstrated bedaquiline Minimum Inhibitory Concentration (MIC) 99 was between 0.01-0.1 μg/ml against a variety of Mycobacterium tuberculosis isolates, regardless of resistance to other conventionally used anti-TB drugs (Andries, K., Verhasselt, P., Guillemont, J., Göhlmann, H W H., Neefs, J M., Winkler, H., Van Gestel, J., Timmerman, P., Zhu, M., Lee, E., Williams, P., de Chaffoy, D., Huitric, E., Hoffner, S., Cambau, E., Truffot-Pernot, C., Lounis, N. and Jarlier, V., “A diarylquinoline drug active on the ATP synthase of Mycobacterium tuberculosis”, Science (2005), 307: pp. 223-227. These results were replicated using a standardized broth-dilution assay, and demonstrated MIC99 ranges between 0.015-0.12 μg/ml against Mycobacterium tuberculosis H37Rv (Kaniga, K., Cirillo, D M., Hoffner, Ismail, N A., Kaur, D., Louni s, N., Metchock, B., Pfyffer, G E. and Venter, A., “A Multilaboratory, Multicountry Study To Determine Bedaquiline MIC Quality Control Ranges for Phenotypic Drug Susceptibility Testing”, Journal of Clinical Microbiology (2016), 54(12): pp. 2956-2962. Interestingly, this activity extends to other mycobacteria, including both M. avium and M. abscessus, with MIC99 of 0.01-0.03 μg/ml and 0.25-0.5 μg/ml respectively. This activity has been translated to in vivo models, improving bacterial clearance in models of M. tuberculosis and M. abscessus infection (Obregon-Henao, A., Arnett, K A., Henao-Tamayo, M., Massoudi, L., Creissen, E., Andries, K., Lenaerts, A J. And Ordway, D J., “Susceptibility of Mycobacterium abscessus to Antimycobacterial Drugs in Preclinical Models”, Antimicrobial Agents and Chemotherapy (2015), 59(11): pp. 6904-6912; Tasneen, R., Li, S Y., Peloquin, C A., Taylor, D., Williams, K N., Andries, K., Mdluli, K E. and Nuermberger, E L., “Sterilizing Activity of Novel TMC207- and PA-824-Containing Regimens in a Murine Model of Tuberculosis”, Antimicrobial Agents and Chemotherapy (2011), 55(12); pp. 5485-5492). The sterilizing activity of BDQ can also work synergistically with numerous anti-TB drugs, such as ethambutol, pyrazinamide, linezolid, and clofazimine (Obregon-Henao, A., Arnett, K A., Henao-Tamayo, M., Massoudi, L., Creissen, E., Andries, K., Lenaerts, A J. And Ordway, D J., “Susceptibility of Mycobacterium abscessus to Antimycobacterial Drugs in Preclinical Models”, Antimicrobial Agents and Chemotherapy (2015), 59(11): pp. 6904-6912; Reddy, V M., Einck, L., Andries, K. and Nacy, C A., “In Vitro Interactions between New Antitubercular Drug Candidates SQ109 and TMC207”, Antimicrobial Agents and Chemotherapy (2010), 54(7): pp. 2840-2846; Tasneen, R., Williams, K., Amoabeng, O., Minkowski, A., Mdluli, K E., Upton, A M. and Nuermberger, E L., “Contribution of the Nitroimidazoles PA-824 and TBA-354 to the Activity of Novel Regimens in Murine Models of Tuberculosis”, Antimicrobial Agents and Chemotherapy (2015), 59(1): pp. 129-135; Lamprecht, D A., Finin, P M., Rahman, A., Cumming, B M., Russell, S L., Jonnala, S R., Adamson, J H. and Steyn, A J C., “Turning the respiratory flexibility of Mycobacterium tuberculosis against itself”, Nature Communications (2016): DOI: 10.1038/ncomms123

Table 1 shows MIC's of Bedaquline against different Mycobacteria (μg/ml) (Soni, I., De Groote, M A., Dasgupta, A. and Chopra, S., “Challenges facing the drug discovery pipeline for non-tuberculous mycobacteria”, Journal of Medical Microbiology (2016), 65: pp. 1-8).

TABLE 1

| Mycobacterium sp. | MIC (μg/ml) |
|---|---|
| M. abscessus | 0.25 |
| M. ulcerans | 0.5 |
| M. intracellulare | 0.010 |
| M. marinum | 0.003 |
| M. smeqmatis mc2 155 | 0.12 |
| M. avium | 0.03-0.13 |
| M. kansasii | 0.03 |
| M. fortuitum | 0.13-0.25 |
| M. intracellulare | 0.03-0.25 |
| M. chelonae | 0.06-0.5 |
| M. maqeritense | 0.03 |
| M. phlei | 0.03-0.13 |
| M. vaccae | 0.03 |
| M. malmoense | 0.50 |
| M. qordonae | 0.03 |
| M. simiae | 0.03 |
| M. scrofulaceum | 0.03 |
| M. hiberniae | 0.03 |
| Drug-susceptible M. tuberculosis | 0.06 |
| MOR M. tuberculosis | 0.06 |

Currently, BDQ is administered orally, where it reaches its maximal plasma concentration 4-6 hours after administration (Andries, K., Verhasselt, P., Guillemont, J., Göhlmann, H W H., Neefs, J M., Winkler, H., Van Gestel, J., Timmerman, P., Zhu, M., Lee, E., Williams, P., de Chaffoy, D., Huitric, E., Hoffner, S., Cambau, E., Truffot-Pernot, C., Lounis, N. and Jarlier, V., “A diarylquinoline drug active on the ATP synthase of Mycobacterium tuberculosis”, Science (2005), 307: pp. 223-227). These serum concentrations are proportional to dosage, and BDQ biological activity is concentration dependent, with Area Under the Curve (AUG) measurements being the main predictor of drug efficacy (Rouan, M C., Lounis, N., Gevers, T., Dillen, L., Gilissen, R., Raoof, A. and Andries, K., “Pharmacokinetics and Pharmacodynamics of TMC207 and Its N-Desmethyl Metabolite in a Murine Model of Tuberculosis”, Antimicrobial Agents and Chemotherapy (2012), 56(3): pp. 1444-1451). Food intake with BDQ has been demonstrated to improve bioavailability, increasing the drug AUG 2-4 fold relative to fasted conditions (Diacon, A H., Pym, A., Grobusch, M., Patientia, R., Rustomjee, R., Page-Shipp, L., Pistorius, C., Krause, R., Bogoshi, M., Churchyard, G., Venter, A., Allen, J., Palomino, J C., De Marez, T., van Heeswijk, R P G., Lounis, N., Meyvisch, P., Verbeeck, J., Parys, W., de Beule, K., Andries, K. and Mc Neeley, D F., “The Diarylquinoline TMC207 for Multidrug-Resistant Tuberculosis”, The New England Journal of Medicine (2009), 360: pp. 2397-2405; van Heeswijk, R P G., Dannemann, B. and Hoetelmans, R M W., “Bedaquiline: a review of human pharmacokinetics and drug-drug interactions”, Journal of Antimicrobial Chemotherapy (2014), 69: pp 0.2310-2318).

Upon administration, bedaquiline has shown preferential tissue accumulation into the lungs and spleen, and high binding to plasma proteins in serum (Andries, K., Verhasselt, P., Guillemont, J., Göhlmann, H W H., Neefs, J M., Winkler, H., Van Gestel, J., Timmerman, P., Zhu, M., Lee, E., Williams, P., de Chaffoy, D., Huitric, E., Hoffner, S., Cambau, E., Truffot-Pernot, C., Lounis, N. and Jarlier, V., “A diarylquinoline drug active on the ATP synthase of Mycobacterium tuberculosis”, Science (2005), 307: pp. 223-227). In Phase II clinical trials of drug susceptible TB, BDQ was measured at Cₘₐₓ 5 μg/ml in the sputum after 7 day treatment of 400 mg, comparable to serum concentrations observed in the same treatment regimen (Rustomjee, R., Diacon, A H., Allen, J., Venter, A., Reddy, C., Patientia, R F., Mthiyane, T C P., De Marez, T., van Heeswijk, R., Kerstens, R., Koul, A., De Beule, K., Donald, P R. and McNeeley, D F., “Early Bactericidal Activity and Pharmacokinetics of the Diarylquinoline TMC207 in Treatment of Pulmonary Tuberculosis”, Antimicrobial Agents and Chemotherapy (2008), 52(8): pp. 2831-2835; Lounis, N., Gevers, T., Van Den Berg, J. and Andries, K., “Impact of the Interaction of R207910 with Rifampin on the Treatment of Tuberculosis Studied in the Mouse Model”, Antimicrobial Agents and Chemotherapy (2008), 52(10): pp. 3568-3572. This high tissue penetration, along with an extensive tissue half life, leads to a long effective half-life of 24 hours, and extended terminal half life of 5.5 months (Andries, K., Verhasselt, P., Guillemont, J., Göhlmann, H W H., Neefs, J M., Winkler, H., Van Gestel, J., Timmerman, P., Zhu, M., Lee, E., Williams, P., de Chaffoy, D., Huitric, E., Hoffner, S., Cambau, E., Truffot-Pernot, C., Lounis, N. and Jarlier, V., “A diarylquinoline drug active on the ATP synthase of Mycobacterium tuberculosis”, Science (2005), 307: pp. 223-227; Janssen Pharmaceutical Companies, Briefing Document “TMC207 (bedaquiline) Treatment of Patient with MOR-TB”, FDA Anti-Infective Drugs Advisory Committee Meeting (Nov. 28, 2012) pp. 1-253).

Despite the benefits of BDQ addition to mycobacterial treatment regimens, there are adverse events related to treatment. The most common tissues affected include hepatic and cardiac tissue, with QT-interval elongation and electrical rhythm disturbances most common for the latter (Kwon, Y S. And Koh, W J., “Synthetic investigational new drugs for the treatment of tuberculosis”, Expert Opinion on investigational Drugs (2016), 25(2): pp. 183-193; Goulooze, S C., Cohen, A F. and Rissmann, R., “Bedaquiline”, British Journal of Clinical Pharmacology (2015), 80(2): pp. 182-184; Kakkar, A K. and Dahiya, N., “Bedaquiline for the treatment of resistant tuberculosis: promises and pitfalls.”, Tuberculosis (2014), 94(4): pp. 357-362). Of concern, increased mortality rates are also associated with current BDQ therapy, although death was attributed to respiratory disorders, and not BDQ toxicity (Diacon, A H., Pym, A., Grobusch, M P., de las Rios, J M., Gotuzzo, E., Vasilyeva, I., Leimane, V., Andries, K., Bakare, N., De Marez, T., Haxaire-Theeuwes, M., Lounis, N., Meyvisch, P., De Paepe, E. and van Heeswijk, R P G., “Multidrug-Resistant Tuberculosis and Culture Conversion with Bedaquiline”, The New England Journal of Medicine (2014), 317: pp. 723-732; Mingote, L R., Namutamba, D Apina, F., Barnabas, N., Contreras, C., Elnour, T., Frick, M W., Lee, C., Seaworth, B., Shelly, D., Skipper, N. and dos Santos Filho, E T., “The use of bedaquiline in regimens to treat drug-resistant and drug-susceptible tuberculosis: a perspective from tuberculosis-affected communities”, Lancet (2015), 385: pp. 477-479).

Additional concerns have arisen regarding BDQ drug-drug interactions, with particular concern over interactions between BDQ and anti-TB drugs, as well as anti-viral agents for the treatment of human immunodeficiency virus (HIV) (which has a high coinfection rate with TB) (http://apps.who.int/iris/bitstream/10665/191102/1;9879241566509 eng.pdf) (last accessed Jan. 4, 2018)). In fact, co-treatment of BDQ with rifamycin-group antibiotics has been demonstrated to reduce BDQ AUC by up to 59%, due to rifampicin's ability to induce CYP enzyme activity (van Heeswijk, R P G., Dannemann, B. and Hoetelmans, R M W., “Bedaquiline: a review of human pharmacokinetics and drug-drug interactions”, Journal of Antimicrobial Chemotherapy (2014), 69: pp. 2310-2318). Similar interactions with numerous anti-virals, although coadministration of BDQ with lopinavir/ritonavir lead to increased BDQ concentrations, instead decreasing the anti-viral concentrations. However, it should be noted that many of these studies are single dose interactions, and more prolonged treatment studies are needed due to the long residence time of BDQ.

The advantages of pulmonary delivery of antimycobacterial therapy have been summarized by Das (Das, S., Tucker, I., and Stewart, P., “Inhaled Dry Powder Combinations for Treating Tuberculosis”, Current Drug Delivery (2015), 12: pp. 26-39), for example, as follows:

First, the concentration of drug at the lung is higher compared to intramuscular administration. This higher drug concentration helps to prevent biofilm formation and reduces the risk of drug-resistance.

The frequency of administration can be reduced since the drug remains in the lung for a longer period of time than by intramuscular and intravenous administration.

A reduced dose of drug is required for pulmonary delivery compared to oral administration. Reduced toxicity is associated with the reduced amount of drug in the body.

Improved patient compliance is expected due to reduction of dose, frequency and duration of treatment.

The uptake of drug-microparticles by alveolar macrophages can reverse the “alternative activation” and trigger the bactericidal responses.

Pulmonary delivery is suitable for delivery of drugs for which the optimal drug concentration at the site of action is difficult to attain.

Pulmonary delivery offers advantage for drugs that are poorly water soluble and difficult to formulate for injection.

The pulmonary route is advantageous in that it avoids injections for those injectable drugs that require frequent administration for a long time.

The decomposition of drugs by gastrointestinal environment can be avoided by pulmonary administration. For example, rifampicin, which is degradable by the acidic environment of stomach in the presence of isoniazid, can be administered via pulmonary route.

Finally, and relatedly, pulmonary administration allows the avoidance of hepatic first pass metabolism.

Many of these potential advantages can be achieved by pulmonary administration of bedaquiline as opposed to oral administration of bedaquiline. Recall, for example, the low solubility of bedaquiline in water. During current oral treatment (400 mg once daily for 2 weeks followed by 200 mg 3 times per week for 22 weeks), after ingestion of the pill, bedaquiline must first dissolve in the stomach fluid, then diffuse into the blood. High penetration rates to the spleen and binding with plasma proteins in the serum decrease the drug available to enter the lungs. After circulation to the lungs, the drug must diffuse into the lung tissue, then into the macrophages where the mycobacteria reside. Because of the extremely low solubiliy of bedaquiline, this is a very inefficient system, and much of bedaquiline is excreted with feces. By delivering bedaquiline directly to the lung periphery, it can be directly ingested by macrophages and act on mycobacteria. Bypassing the inefficient oral delivery route means that the pulmonary dose will be lower than the oral dose (10 mg to 100 mg, depending on the particulars of inhaled administration).

Bedaquiline has a very long half life in tissues, more than 5 months. By depositing bedaquiline directly in the lung tissue, treatment durations can be decreased compared to oral therapy.

Accordingly, the use of an aerosolized administration of bedaquiline in patients with multi-drug resistant tuberculosis, or extensively drug resistant tuberculosis infections should further improve patient treatment outcomes, and may shorten the duration of current treatment regimens.

The group of nontuberculous mycobacteria (NTM), formerly called atypical or ubiquitous mycobacteria, contains over 150 species. NTM can be found ubiquitously in nature and show a broad diversity. They can be detected in soil, ground and drinking water as well as in food like pasteurized milk or cheese. In general, NTM are considered to be less pathogenic. Nevertheless, they can cause severe illness in humans, especially in immune compromised persons or those who suffer from previous pulmonary diseases. Currently NTM are classified according to their growth rate and are divided into slow-growing (SGM) and rapid-growing (RGM) mycobacteria.

The slow growing Mycobacterium avium complex (MAC) comprises the species Mycobacterium avium, Mycobacterium chimaera and Mycobacterium intracellulare that are among the most important and most frequent pathogenic NTM. Just like Mycobacterium kansasii, Mycobaceterium malmoense, Mycobacterium xenopi, Mycobacterium, simiae, Mycobacterium abscessus, Mycobacterium gordonae, Mycobacterium fortuitum, and Mycobacterium chelonae, they mostly cause pulmonary infections. Mycobacterium marinum is responsible for skin and soft tissue infections like aquarium granuloma.

In particular, RGM cause serious, life-threatening chronic lung diseases and are responsible for disseminated and often fatal infections. Infections are typically caused by contaminated materials and invasive procedures involving catheters, non-sterile surgical procedures or injections and implantations of foreign bodies. Exposure to shower heads and jacuzzis has also been reported as risks for infections. NTM typically cause opportunistic infections in patients with chronic pulmonary diseases such as chronic obstructive pulmonary disease (COPD), cystic fibrosis (CF), and other immune compromised patients.

In recent years, the rapidly growing (RGM) Mycobacterium abscessus group strains (Mycobacterium abscessus complex, MABSC) comprising the subspecies Mycobacterium abscessus subsp. abscessus (M. a. abscessus), Mycobacterium abcessus bolletii. and Mycobacterium abscessus massiliense have emerged as important human pathogens and are associated with significantly higher fatality rates than any other RGM.

Mycobacterium abscessus infection in CF patients are particularly problematic, as it results in enhanced pulmonary destruction and is often impossible to treat with failure rates as high as 60-66%. (see, for example, Obregon-Henao A et al, Antimicrobial Agents and Chemotherapy, November 2015, Vol 59, No 11, p. 6904-6912; Qvist, T., Pressler, T., Hoiby, N. and Katzenstein, T L., “Shifting paradigms of nontuberculous mycobacteria in cystic fibrosis”, Respiratory Research (2014), 15(1): pp 0.41-47).

Human infection with NTM became of greater relevance with the emergence of the human acquired immune deficiency syndrome (AIDS) pandemic. Mycobacteria from Mycobacterium avium complex (MAC) were identified as the major cause of opportunistic infections in patients infected with the human immunodeficiency virus (HIV).

Several species of NTM are known to form biofilms. Biofilms are microcolonies of bacteria embedded in the extracellular matrix that provide stability and resistance to human immune mechanisms. In recent years, some species of NTM have been shown to form biofilms that enhance resistance to disinfectants and antimicrobial agents. Biofilm assembly proceeds through several phases, including reversible attachment, irreversible attachment, biofilm formation via bacterial aggregation, organization, and signaling, and finally dispersion. During this process, bacteria develop a matrix containing extracellular polymeric substances (EPS), such as polysaccharides, lipids and nucleic acids, to form a complex three-dimensional structure (see, for example, Sousa S. et al., International Journal of Mycobacteriology 4 (2015), 36-43). Specifically, mycobacterial EPS differ in nature from other biofilms, as mycobacteria do not produce exopolysaccharides (see, for example, Zambrano M M, Kolter R. Mycobacterial biofilms: a greasy way to hold it together. Cell. 2005). Mycobacterial biofilms vary between species, but can contain mycolic acids, glycopeptidolipids, mycolyl-diacylglycerols, lipooligosaccharides, lipopeptides, and extracellular DNA (Overview and original research from: Rose S J, Babrak L M, Bermudez L E (2015) Mycobacterium avium Possesses Extracellular DNA that Contributes to Biofilm Formation, Structural Integrity, and Tolerance to Antibiotics, PLoS ONE). The assembly in biofilms is known to enhance resistance to antimicrobial agents (see, for example, Faria S. et al., Journal of Pathogens, Vol 2015, Article ID 809014).

Delivery of aerosolized liposomal amikacin/inhaled amikacin solution nebulized by a jet nebulizer as a novel approach for treatment of NTM pulmonary infection has been suggested (Rose S. et al, 2014, PLoS ONE, Volume 9, Issue 9, e108703, and Olivier K. et al, Ann Am Thorac Soc Vol 11, No 1, pp. 30-35) as well as inhalation of anti-TB drugs dry powder microparticles for pulmonary delivery (Cholo M et al., J Antimicrob Chemother. 2012 February; 67(2):290-8 and Fourie B. and Nettey 0., 2015 Inhalation Magazine, Verma 2013 Antimicrob Agents Chemother).

Multiple combination regimens with inhaled amikacin following initial treatment with parenteral aminoglycosides, tigecycline and other promising oral antibiotics such as linezolid, delamanid, and bedaquiline, and surgical intervention in selected cases have shown promising results in the treatment of NTM lung disease (Lu Ryu et al., Tuberc Respir Dis 2016; 79:74-84). However, the increasing incidence and prevalence of NTM infections, in particular NTM lung disease and the limited treatment options necessitate the development of novel dosage forms/pharmaceutical formulations and combinations enhancing the bioavailability of the currently used antibiotics such as bedaquiline. Inhalation may enhance efficacy and reduce adverse effects compared to oral and parenteral therapies.

Synergy has been shown with combinations of bedaquiline and clofazimine used against Mycobacterium tuberculosis (see, for example, Cokol, M. et al., “Efficient Measurement and factorization of high-order drug interactions in Mycobacterium tuberculosis”, Sciences Advances 2017:3:e170881, 11 Oct. 2017).

Bedaquiline has also been shown to have an additive effect with amikacin (see, for example, https://www.escmid.org/escmid_pulications/escmid_elibrary/material/?mid=42441).

The low solubility of bedaquiline in water results in low oral bioavailability and high microbial resistance and also requires specific techniques to solubilise and stabilize the drug for formulation in liquid aqueous carriers such as for aerosolization by nebulizers in order to obtain lower lung deposition of the aerosol particles.

### SUMMARY OF THE INVENTION

The present invention provides bedaquiline in the form of a suspension compatible with an appropriate nebulizer, or as a dry powder compatible with a dry powder inhaler, which generate the suitable aerosol particles to provide significantly increased delivery of the aerosolized bedaquiline into the lower lung (i.e. to the bronchi, bronchioli, and alveoli of the central and lower peripheral lungs.

The invention provides for an aerosol having aerosol particles of sizes that facilitate delivery to the alveoli and bronchiole, thereby substantially enhancing therapeutic efficacy. A suitable aerodynamic particle size for targeting the alveoli and bronchiole is between 1 and 5 μm. Aerosol particles larger than that are selectively deposited in the upper lungs, namely bronchi and trachea and in the mouth and throat, i.e. oropharyngeal area. Accordingly, the inhalation device is adapted to produce an aerosol having a mass median aerodynamic diameter (MMAD) in the range from about 1 to about 5 μm, and preferably in the range from about 1 to about 3 μm. In a further embodiment, the particle size distribution is narrow and has a geometric standard deviation (GSD) of less than about 2.5.

Aerosol dosage, formulations and delivery systems may be selected for a particular therapeutic application, as described, for example in Gonda, I. “Aerosols for delivery of therapeutic and diagnostic agents to the respiratory tract”, Critical Reviews in Therapeutic Drug Carrier Systems, 6, 273-314 (1990), and Moren, “Aerosol dosage forms and formulations”, Aerosols in Medicine, Principles, Diagnosis and Therapy, Moren, et al., Eds. Elsevier, Amsterdam, 1985.

### DETAILED DESCRIPTION OF THE INVENTION

The present invention is based on the discovery that by pulmonary administration of bedaquiline in the form of an aerosol, lower (i.e. deeper) lung deposition of the active agent can be achieved, thereby significantly increasing the bioavailability of this extremely hydrophobic BCS class II agent, which results in significantly increased therapeutic efficacy coupled with reduced systemic side effects.

In another aspect, this finding leads to the provision of an improved antibiotic therapy for infections caused by mycobacteria and gram-positive bacteria, in particular of pulmonary infections with NTM, such as opportunistic infections in CF, COPD and immune compromised patients such as HIV patients.

The present invention, moreover, aims at overcoming systemic side effects of established oral treatment regimens for pulmonary infections with gram positive bacteria, in particular TB and NTM infections of the lungs as well as at the reduction of dose and of duration of treatment with bedaquiline.

It is understood by the person of skill in the art that the present application also discloses each and any combination of the individual features disclosed herein.

### Definitions

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobionic acid, tartaric acid, benzoic acid, cinnamic acid, mandelic acid, methanesulfonic acid, ethanesulfonic acid, p-toluenesulfonic acid, salicylic acid, and the like.

Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsufuric acid, naphthalene-1,5-disulfonic acid, naphthalene-2-sulfonic acid, and saccharin.

In accordance with the present invention, apart from the free base, the use of the fumaric acid, sulfuric acid, tartaric acid, citric acid, phosphoric acid salts of bedaquiline, and in particular the fumaric acid salt of bedaquiline is preferred.

As used herein, the term, “pharmaceutically acceptable derivative” of a compound is, for example, a prodrug of said compound. In general, a prodrug is a derivative of a compound which, upon administration, is capable of providing the active form of the compound. Such derivatives, for example, may be an ester or amide of a carboxyl group, an carboxyl ester of a hydroxyl group, or a phosphate ester of a hydroxyl group.

By “patient” is meant a mammal, preferably a human, in need of the prophylaxis and/or the treatments as described herein.

By “therapeutically effective amount”, “therapeutically effective dose”, or “pharmaceutically effective amount” is meant an amount of bedaquiline, as disclosed for this invention, which has a therapeutic effect in a patient. The doses of bedaquiline which are useful in treatment are therapeutically effective amounts. Thus, as used herein, a therapeutically effective amount means those amounts of bedaquiline which produce the desired therapeutic effect as judged by clinical trial results and/or model animal infection studies.

The amount of the bedaquiline and daily dose can be routinely determined by one of skill in the art, and will vary, depending on several factors, such as the particular microbial strain involved. This amount can further depend upon the patient's height, weight, sex, age and medical history. For prophylactic treatments, a therapeutically effective amount is that amount which would be effective to prevent a microbial infection.

A “therapeutic effect” relieves, to some extent, one or more of the symptoms of the infection, and includes curing an infection. “Curing” means that the symptoms of active infection are eliminated, including the total or substantial elimination of excessive members of viable microbe of those involved in the infection to a point at or below the threshold of detection by traditional measurements. However, certain long-term or permanent effect of the infection may exist even after a cure is obtained (such as extensive tissue damage). As used herein, a “therapeutic effect” is defined as a statistically significant reduction in bacterial load in a host, emergence of resistance, or improvement in infection symptoms as measured by human clinical results or animal studies.

“Treat”, “treatment”, or “treating” as used herein refers to administering a pharmaceutical composition/formulation to a patient for prophylactic and/or therapeutic purposes.

The term “prophylactic treatment” or “prophylaxis” refers to treating a patient who is not yet infected, but who is susceptible to, or otherwise at risk of, a particular infection. The term “therapeutic treatment” refers to administering treatment to a patient already suffering from an infection. Thus, in preferred embodiments, treating is the administration to a mammal (either for therapeutic or prophylactic purposes) of therapeutically effective amounts of bedaquiline.

Unless stated otherwise herein, the term “inhalation” is meant to refer to oral inhalation into the lungs.

Unless stated otherwise herein, the term “infection” as used herein is meant to refer to pulmonary infections.

Unless otherwise stated, the term “substantially” when used to refer to the purity of a compound, indicates a purity of compound of 95% or greater purity.

Unless otherwise stated, the term “appropriate particle size” refers to a particle size of bedaquiline in a composition or as provided by a pharmaceutical combination that provides the desired therapeutic effect when administered to a patient.

Unless otherwise stated, the term “appropriate concentration” refers to a concentration of a component in a composition or pharmaceutical combination which provides a pharmaceutically acceptable composition or combination.

### Pharmaceutical Compositions and Combinations

The following water grades are particularly applicable to the present invention: sterile purified water, sterile water for injection, sterile water for irrigation, sterile water for inhalation (USP) and corresponding water grades in accordance with e.g. European Pharmacopoeia or National Formulary.

Aqueous electrolyte solutions as used in accordance with the present invention as the aqueous liquid carrier may further comprise sodium chloride, potassium chloride, lithium chloride, magnesium chloride, calcium chloride or mixtures thereof.

The aqueous liquid carrier is preferably isotonic saline solution (0.9% NaCl corresponding to approximately 150 mM NaCl, preferably 154 mM NaCl).

Accordingly, in an embodiment of the present invention, a pharmaceutical composition is provided comprising: (a) a therapeutically effective dose of bedaquiline or a pharmaceutically acceptable derivative or salt thereof; (b) a nonionic surfactant with an Hydrophilic-Lipophilic Balance value of greater than 10; and (c) an aqueous liquid carrier selected from water, isotonic saline, buffered saline and aqueous electrolyte solutions, wherein the bedaquiline or the pharmaceutically acceptable derivative or salt thereof is provided in the form of particles in a suspension, and wherein the bedaquiline particles, or the particles of the pharmaceutically acceptable salt of bedaquiline, have a median size of less than 5 μm and a D90 of less than 6.5 μm. In another embodiment, the particles of bedaquiline, or the pharmaceutically acceptable salt thereof, have a median size of less than 2 μm and a D90 of less than 3 μm.

In a further embodiment of the present invention, a pharmaceutical composition is provided comprising (a) a therapeutically effective dose of bedaquiline; (b) a nonionic surfactant with an Hydrophilic-Lipophilic Balance value of greater than 10; and (c) an aqueous liquid carrier selected from water, isotonic saline, buffered saline and aqueous electrolyte solutions, wherein the bedaquiline is provided in the form of particles in a suspension, and wherein the bedaquiline particles have a median size of less than 5 μm and a D90 of less than 6.5 μm. In a further embodiment, the bedaquiline particles have a median size of less than 2 μm and a D90 of less than 3 μm.

In another embodiment of the present invention, a pharmaceutical composition according the any of the embodiments described above, wherein the nonionic surfactant is selected from polysorbate 20 (for example Tween® 20, polysorbate 60 (for example Tween® 60), polysorbate 80 (for example Tween® 80), stearyl alcohol, a polyethylene glycol derivative of hydrogenated castor oil with an Hydrophilic-Lipophilic Balance value of 14 to 16 (for example Cremophor® RH 40), a polyethylene glycol derivative of hydrogenated castor oil with an Hydrophilic-Lipophilic Balance value of 15 to 17 (for example Cremophor® RH 60), sorbitan monolaurate (for example Span® 20), sorbitan monopalmitate (for example Span® 40), sorbitan monostearate (for example Span® 60), polyoxyethylene (20) oleyl ether (for example Brij® 020), polyoxyethylene (20) cetyl ether (for example Brij® 58), polyoxyethylene (10) cetyl ether (for example Brij® C10), polyoxyethylene (10) oleyl ether (for example Brij® 010), polyoxyethylene (100) stearyl ether (for example Brij® S100), polyoxyethylene (10) stearyl ether (for example Brij® S10), polyoxyethylene (20) stearyl ether (for example Brij® S20), polyoxyethylene (4) lauryl ether (for example Brij® L4), polyoxyethylene (20) cetyl ether (for example Brij® 93), polyoxyethylene (2) cetyl ether (for example Brij® S2), caprylocaproyl polyoxyl-8 glyceride (for example Labrasol®), polyethylene glycol (20) stearate (for example Myrj™ 49), polyethylene glycol (40) stearate (for example Myrj™ S40), polyethylene glycol (100) stearate (for example Myrj™ S100), polyethylene glycol (8) stearate (for example Myrj™ S8), and polyoxyl 40 stearate (for example Myrj™ 52), and mixtures thereof.

In a preferred embodiment of the invention, a pharmaceutical composition is provided according to any of the embodiments described above, wherein the non-ionic surfactant is polysorbate 80, and wherein the aqueous liquid carrier is distilled water, hypertonic saline, or isotonic saline. In another preferred embodiment the hypertonic saline is from 1% to 7% (weight/volume) sodium chloride. In another preferred embodiment, the non-ionic surfactant is ultrapure polysorbate 80 (for example, NOF Corporation Polysorbate 80 (Hx2)), and the aqueous liquid carrier is isotonic saline.

In another embodiment of the present invention, a pharmaceutical composition according to any of the composition embodiments described above, wherein the osmolality of the composition is in the range of 200-700 mOsm/kg. In a preferred embodiment the osmolality of the composition is in the range of 300-400 mOsm/kg.

In a further embodiment of the present invention, a pharmaceutical composition according to any of the embodiments described above, is provided wherein the concentration of nonionic surfactant is in the range of 0.001% to 5% (v/v) of the total composition and the amount of bedaquiline is in the range of 0.1% to 20% (w/v) of the total composition.

In a further embodiment of the present invention, a pharmaceutical composition according to any of the composition embodiments described above, prepared by a process comprising the following steps: (1) homogenization of a suspension of bedaquiline, the nonionic surfactant and water to obtain a suspension comprising bedaquiline of an appropriate particle size, (2) adjusting the pH of the suspension resulting from (1) to a pH of between pH 5.5 and pH 7.5, (3) adjusting the sodium chloride concentration to an appropriate concentration and (4) adjusting the osmolality to an appropriate level. In a preferred embodiment, the pH is adjusted to 6.5, and the sodium chloride concentration is 154 mM sodium chloride. In another preferred embodiment, the homogenization in step (1) is carried out by high pressure homogenization, high shear homogenization, wet milling, ultrasonic homogenization, or a combination of such processes. In another preferred embodiment of the invention, the homogenization of bedaquiline is carried out in multiple steps of homogenization.

In another embodiment of the present invention, a pharmaceutical composition according to any of the composition embodiments described above is provided, prepared by a process comprising the following steps: (1) homogenization of a suspension of bedaquiline and a non-aqueous liquid to obtain a suspension comprising bedaquiline of an appropriate particle size, (2) isolation of the bedaquiline, (3) addition of the bedaquiline to the nonionic surfactant and water, (4) adjusting the pH of the suspension resulting from (3) to a pH of between pH 5.5 and pH 7.5, and (5) adjusting the sodium chloride concentration to an appropriate concentration. In a preferred embodiment, the pH is adjusted to 6.5, and the sodium chloride concentration is 154 mM sodium chloride. In another preferred embodiment, the homogenization in step (1) is carried out by high pressure homogenization, high shear homogenization, wet milling, ultrasonic homogenization, or a combination of such processes. In another preferred embodiment of the invention, the homogenization of bedaquiline is carried out in multiple steps of homogenization.

In another embodiment, a pharmaceutical composition according to any of the composition embodiments as described above is provided, prepared by a process comprising the following steps: (1) micronization of bedaquiline to obtain bedaquiline of an appropriate particle size, (2) addition of the bedaquiline to the nonionic surfactant and water, (3) adjusting the pH of the suspension resulting from (2) to a pH of between pH 5.5 and pH 7.5, and (4) adjusting the sodium chloride concentration to an appropriate concentration. In a preferred embodiment, the pH is adjusted to 6.5, and the sodium chloride concentration is adjusted to 154 mM sodium chloride. In another preferred embodiment, the micronization of the bedaquiline is carried out by jet milling, spray drying, ball milling, or super critical fluids processing. In another preferred embodiment of the invention, the micronization of bedaquiline is carried out in multiple steps of homogenization.

In a further embodiment, a pharmaceutical composition according to any of claims the composition embodiments described above is provided, prepared by a process comprising homogenization of a suspension of bedaquiline in the nonionic surfactant, water containing an appropriate concentration of sodium chloride, and which has been adjusted to a pH of between pH 5.5 and pH 7.5, to obtain bedaquiline of an appropriate particle size. In a preferred embodiment, the pH is adjusted to 6.5, and the sodium chloride concentration is adjusted to 154 mM sodium chloride. In another preferred embodiment, the homogenization in step (1) is carried out by high pressure homogenization, high shear homogenization, wet milling, ultrasonic homogenization, or a combination of such processes. In another preferred embodiment of the invention, the homogenization of bedaquiline is carried out in multiple steps of homogenization.

In another embodiment of the invention a composition prepared by any of the process embodiments described above is provided, wherein the appropriate particle size of the bedaquiline are particles having a mean size of less than 5 μm and D90 of less than 6.5 μm. In a preferred embodiment the appropriate particle size of the bedaquiline are particles having a mean size of less than 2 μm and D90 of less than 3 μm.

In another embodiment of the invention, a pharmaceutical combination in the form of an aerosol for inhalation is provided, prepared by aerosolization of the any of the composition embodiments, or any of the compositions prepared by any of the process embodiments described above, by a nebulizing device selected from an ultrasonic nebulizer, an electron spray nebulizer, a vibrating membrane nebulizer, a jet nebulizer and a mechanical soft mist inhaler, and wherein the aerosol particles produced by the nebulizing device have a mass median aerodynamic diameter of 1 to 5 μm. In another embodiment the aerosol for inhalation is for lower lung deposition. In a further embodiment, the nebulizing device exhibits an output rate of 0.1-1.0 ml/min. In another embodiment, the total inhalation volume is between 1 ml and 5 ml. In another embodiment, the pharmaceutical combination is for use in the treatment and/or prophylaxis of pulmonary infections caused by mycobacteria or other gram positive bacteria. In a further embodiment, the infection is caused by a species of the genus Mycobacterium selected from nontuberculous mycobacteria and Mycobacterium tuberculosis complex, and a combination thereof. In a further embodiment, the nontuberculous mycobacteria is selected from Mycobacterium avium, Mycobacterium intracellulare, Mycobacterium abscessus, and Mycobacterium leprae, and a combination thereof. In another embodiment, the infection is an opportunistic infection, selected from MAC pulmonary disease and nontuberculosis infection, in a patient with cystic fibrosis, chronic obstructive pulmonary disease or acquired immune deficiency syndrome. In another embodiment, the infection is an opportunistic nontuberculosis mycobacteria infection in a patient with cystic fibrosis. In a further embodiment, a pharmaceutical combination is provided which is to be used as described above, wherein the pharmaceutical combination is used to administer before, simultaneously or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof, In a further embodiment, the agent is amikacin.

In another embodiment of the invention, any of the composition embodiments, or any of the compositions prepared by any of the process embodiments described above, is used in combination with an agent for dispersing and/or destruction of biofilm, with mucolytic and/or mucoactive agents, and/or agents that reduce biofilm formation selected from nebulized 4-7% hypertonic saline, metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides. In another embodiment, the composition is administered before, simultaneously or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In a further embodiment the agent is clofazimine. In a further embodiment the agent is amikacin.

In another embodiment, a pharmaceutical combination is provided which is to be used in combination with an agent for dispersing and/or destruction of biofilm, with mucolytic and/or mucoactive agents, and/or agents that reduce biofilm formation selected from nebulized 4-7% hypertonic saline, metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides. In a further embodiment, a pharmaceutical combination is provided which is to be used as described above, wherein the pharmaceutical combination is used to administer before, simultaneously or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In a further embodiment, the agent is clofazimine. In a further embodiment the agent is amikacin.

In another embodiment of the invention, a pharmaceutical composition according to any of the composition embodiments, or any of the compositions prepared by any of the process embodiments described above, is provided for use in the treatment and/or prophylaxis of pulmonary infections caused by mycobacteria or other gram positive bacteria. In a further embodiment, the infection is caused by a species of the genus Mycobacterium selected from nontuberculous mycobacteria and Mycobacterium tuberculosis complex, and a combination thereof. In another embodiment, the nontuberculous mycobacteria is selected from Mycobacterium avium, Mycobacterium intracellulare, Mycobacterium abscessus, and Mycobacterium leprae, and a combination thereof. In a further embodiment, the infection is an opportunistic infection, selected from MAC pulmonary disease and nontuberculosis infection, in a patient with cystic fibrosis, chronic obstructive pulmonary disease or acquired immune deficiency syndrome. In another embodiment, the infection is an opportunistic nontuberculosis mycobacteria infection in a patient with cystic fibrosis. In a further embodiment, the pharmaceutical composition for the use described above is administered before, simultaneously or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In a further embodiment the agent is clofazimine. In a further embodiment, the agent is amikacin.

In a further embodiment of the present invention, a system is provided for use in providing antibiotic activity when treating or providing prophylaxis against a pulmonary infection caused by mycobacteria or other gram-positive bacteria, wherein the system comprises: 1) a nebulized pharmaceutical formulation comprising: (a) a therapeutically effective dose of bedaquiline; (b) a nonionic surfactant with an Hydrophilic-Lipophilic Balance value of greater than 10; and (c) an aqueous liquid carrier selected from water, isotonic saline, buffered saline and aqueous electrolyte solutions, and 2) a nebulizer, wherein the bedaquiline is present in the form of a suspension, and wherein the aerosol particles produced by the system have a mass median aerodynamic diameter of 1 to 5 μm.

In a further embodiment, a method of treatment or prophylaxis of a pulmonary infection caused by mycobacteria or other gram positive bacteria is provided, in a patient in need thereof, comprising administering by inhalation a composition according to any of the composition embodiments described above. In a further embodiment, the infection is caused by a species of the genus Mycobacterium selected from nontuberculous mycobacteria and Mycobacterium tuberculosis complex, and a combination thereof. In another embodiment, the nontuberculosis Mycobacterium is selected from Mycobacterium avium, Mycobacterium intracellulare, Mycobacterium abscessus, and Mycobacterium leprae, and a combination thereof. In a further embodiment, the infection is an opportunistic infection, selected from MAC pulmonary disease and nontuberculosis infection, in a patient with cystic fibrosis, chronic obstructive pulmonary disease or acquired immune deficiency syndrome. In a further embodiment the infection is an opportunistic nontuberculosis mycobacteria infection in a patient with cystic fibrosis. In a further embodiment, the composition for inhalation is administered before, simultaneously, or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In a further embodiment, the agent is clofazimine or amikacin. In a further embodiment, the agent is clofazimine.

In another embodiment of the present invention, a process for the preparation of pharmaceutical compositions as described herein is provided, comprising the following steps: (1) homogenization of a suspension of bedaquiline, the non-ionic surfactant and water to obtain a suspension comprising bedaquiline of an appropriate particle size, (2) adjusting the pH of the suspension resulting from (1) to a pH of between pH 5.5 and pH 7.5, (3) adjusting the sodium chloride concentration to an appropriate concentration, and (4) adjusting the osmolality to an appropriate level. In a further embodiment, the pH is adjusted to 6.5, and the sodium chloride concentration is adjusted to 154 mM sodium chloride. In a further embodiment, the homogenization is carried out by high pressure homogenization, wet milling, ultrasonic homogenization, or a combination of such processes. In a further embodiment, the homogenization is carried out in multiple steps of homogenization. In a further embodiment, the appropriate particle size of bedaquiline are particles having a mean size of less than 5 μm and D90 of less than 6.5 μm. In a further embodiment, wherein the appropriate particle size of bedaquiline are particles having a mean size of less than 2 μm and D90 of less than 3 μm.

In another embodiment of the present invention, a process for the preparation of pharmaceutical compositions as described herein is provided, comprising the following steps: (1) homogenization of a suspension of bedaquiline and a non-aqueous liquid to obtain a suspension comprising bedaquiline of the appropriate particle size, (2) isolation of the bedaquiline, (3) addition of the bedaquiline to the nonionic surfactant and water, (4) adjusting the pH of the suspension resulting from (3) to a pH of between pH 5.5 and 7.5, and (5) adjusting the sodium chloride concentration to an appropriate concentration. In a further embodiment, the pH is adjusted to 6.5, and the sodium chloride concentration is adjusted to 154 mM sodium chloride. In a further embodiment, the homogenization is carried out by high pressure homogenization, wet milling, ultrasonic homogenization, or a combination of such processes. In a further embodiment, the homogenization is carried out in multiple steps of homogenization. In a further embodiment, the appropriate particle size of bedaquiline are particles having a mean size of less than 5 μm and D90 of less than μm. In a further embodiment, wherein the appropriate particle size of bedaquiline are particles having a mean size of less than 2 μm and D90 of less than 3 μm.

In another embodiment of the present invention, a process for the preparation of pharmaceutical compositions as described herein is provided, comprising the following steps: (1) micronization of bedaquiline to obtain bedaquiline of an appropriate particle size, (2) addition of the bedaquiline to the nonionic surfactant and water, (3) adjusting the pH of the suspension resulting from (2) to a pH of between pH 5.5 and pH 7.5, and (4) adjusting the sodium chloride concentration to an appropriate concentration. In a further embodiment, the pH is adjusted to 6.5, and the sodium chloride concentration is adjusted to 154 mM sodium chloride. In a further embodiment, the micronization of the bedaquiline is carried out by jet milling, spray drying, ball milling, or super critical fluids processing. In a further embodiment, the micronization of bedaquiline is carried out in multiple steps of micronization. In a further embodiment, the appropriate particle size of bedaquiline are particles having a mean size of less than 5 μm and D90 of less than 6.5 μm. In a further embodiment, wherein the appropriate particle size of bedaquiline are particles having a mean size of less than 2 μm and D90 of less than 3 μm.

In another embodiment of the present invention, a process for the preparation of pharmaceutical compositions as described herein is provided, comprising homogenization of a suspension of bedaquiline in the nonionic surfactant, water containing an appropriate concentration of sodium chloride, and which has been adjusted to a pH of between pH 5.5 and pH 7.5, to obtain bedaquiline of an appropriate particle size. In a further embodiment, the pH is adjusted to 6.5, and the sodium chloride concentration is adjusted to 154 mM sodium chloride. In a further embodiment, the homogenization is carried out by high pressure homogenization, wet milling, ultrasonic homogenization, or a combination of such processes. In a further embodiment, the homogenization is carried out in multiple steps of homogenization. In a further embodiment, the appropriate particle size of bedaquiline are particles having a mean size of less than 5 μm and D90 of less than 6.5 μm. In a further emodiment, wherein the appropriate particle size of bedaquiline are particles having a mean size of less than 2 μm and D90 of less than 3 μm.

In another embodiment of the present invention, a process for the preparation of compositions of the present invention is provided, comprising the following steps: (a) homogenization of a suspension of bedaquiline, the non-ionic surfactant and water to obtain a suspension comprising bedaquiline of an appropriate particle size; (b) adjusting the pH of the resulting suspension a pH of between pH 5.5 and pH 7.5; (c) adjusting the sodium chloride concentration to an appropriate concentration, and (d) adjusting the osmolality to an appropriate level; and wherein steps (b), (c) and (d), may occur in the order of (b), (c), (d); (b), (d), (c); (c), (b), (d); (c), (d), (b); (d), (b), (c); or (d), (c), (b).

In another embodiment of the present invention, a process for the preparation of compositions of the present invention is provided, comprising the following steps: (a) homogenization of a suspension of bedaquiline and a non-aqueous liquid to obtain a suspension comprising bedaquiline of the appropriate particle size; (b) isolation of the bedaquiline; (c) addition of the bedaquiline to the nonionic surfactant and water; (d) adjusting the pH of to resulting suspension to a pH of between pH 5.5 and pH 7.5; and (e) adjusting the sodium chloride concentration to an appropriate concentration; and wherein steps (d) and (e) may occur in the order of (d), (e); or (e), (d).

In another embodiment of the present invention, a process for the preparation of compositions of the present invention is provided, comprising the following steps: (a) micronization of bedaquiline to obtain bedaquiline of an appropriate particle size, and (b) addition of the bedaquiline to the nonionic surfactant, water containing an appropriate concentration of sodium chloride, and which has been adjusted to a pH of between between pH 5.5 and 7.5.

In another embodiment of the present invention, a pharmaceutical composition for dry powder inhalation is provided, comprising bedaquiline of an appropriate particle size, and a physiologically acceptable pharmacologically inert solid carrier, the solid carrier comprising a physiologically acceptable pharmacologically inert excipient, or a mixture of physiologically acceptable pharmacologically inert excipients of appropriate particle size or sizes. In a preferred embodiment of this embodiment, the solid carrier is selected from glucose, arabinose, maltose, saccharose, dextrose and lactose, and combinations thereof. In a further preferred embodiment, the solid carrier is provided in the form of coarse particles having a mass median median diameter of between 50 μm and 500 μm. In still another preferred embodiment, the bedaquiline is provided in the form of finely divided particles having a mass median aerodynamic diameter of less than 5 μm. In still another preferred embodiment, the bedaquiline is provided in the form of finely divided particles having a mass median aerodynamic diameter of between 1 μm and 3 μm.

In a further embodiment of the present invention, a pharmaceutical composition for dry powder inhalation is provided, comprising bedaquiline, or a pharmaceutically acceptable salt or derivative thereof, of an appropriate particle size, and a physiologically acceptable pharmacologically inert excipient, or a mixture of physiologically acceptable pharmacologically inert excipients of appropriate particle size or sizes, wherein the particles of the composition are of a homogeneous composition, wherein the homogeneous particles comprise both bedaquiline and the excipient or excipients. In a preferred embodiment of this embodiment, the particles have a mass median aerodynamic diameter of less than 5 μm. In still another preferred embodiment the particles have a mass median aerodynamic diameter of between 1 μm and 3 μm. In another preferred embodiment of this embodiment, the excipients comprise a phospholipid, or a combination of phospholipids. In still another preferred embodiment, the excipients comprise a salt. In a further preferred embodiment, the excipients comprise an amino acid, or a combination of amino acids. In still another preferred embodiment the excipients comprise a sugar or a combination of sugars.

In another embodiment of the present invention, a pharmaceutical combination is provided, comprising a dry powder inhalation device, the dry powder composition according to any of the dry powder composition embodiments previously described hereinbefore, and a means for introducing the inhalable dry powder composition into the airways of a patient by inhalation. In a preferred embodiment of this embodiment, the dry powder inhalation device is a single dose, or a multi-dose inhaler. In a further preferred embodiment, the dry powder inhalation device is pre-metered or device-metered. In another preferred embodiment, the pharmaceutical combination is for use in the treatment and/or prophylaxis of pulmonary infections caused by mycobacteria or other gram positive bacteria. In another preferred embodiment, the infection is caused by a species of the genus Mycobacterium selected from nontuberculosis mycobacteria and Mycobacterium tuberculosis complex, and a combination thereof. In another preferred embodiment, the nontuberculous bacteria is selected from Mycobacterium avium, Mycobacterium intracellulare, Mycobacterium abscessus, and Mycobacterium leprae, and a combination thereof. In another preferred embodiment, the infection is an opportunistic infection in patients with cystic fibrosis, chronic obstructive pulmonary disease, or AIDS such as Mycobacterium avian complex pulmonary disease or opportunistic nontuberculosis infections associated with cystic fibrosis or chronic obstructive pulmonary disease. In another preferred embodiment the infection is an opportunistic nontuberculosis mycobacteria infection in patients with cystic fibrosis.

In another embodiment of the present invention a pharmaceutical composition according to any of the dry powder composition embodiments described herein is provided for use in the treatment and/or prophylaxis of pulmonary infections caused by mycobacteria or other gram positive bacteria. In a preferred embodiment the pulmonary infection is caused by a species of the genus Mycobacterium selected from nontuberculosis mycbacteria and Mycobacterium tubercuosis complex, and a combination thereof. In a preferred embodiment, the nontuberculosis mycobacteria is selected from Mycobacterium avium, Mycobacterium intracellulare, Mycobacterium abscessus, and Mycobacterium leprae, and a combination thereof. In another preferred embodiment, the infection is an opportunistic infection in patients with cystic fibrosis, chronic obstructive pulmonary disease, or AIDS such as Mycobacterium avian complex pulmonary disease or opportunistic nontuberculosis infections associated with cystic fibrosis or chronic obstructive pulmonary disease. In another preferred embodiment, the infection is an opportunistic nontuberculosis mycobacteria infection in patients with cystic fibrosis.

In another embodiment of the present invention a system is provided for use in providing antibiotic activity when treating or providing prophylaxis against a pulmonary infection caused by mycobacteria or other gram-positive bacteria, wherein the system comprises: 1) a dry powder pharmaceutical formulation comprising a) a therapeutically effective dose of bedaquiline, b) one or more excipients selected from sugars, amino acids, and phospholipids, and combinations thereof, 2) a container for the formulation selected from a capsule or blister package, and 3) a dry powder inhaler, wherein the bedaquiline is present in the form of a dry powder, and wherein the bedaquiline containing particles have a mass median diameter of 1 μm to 5 μm.

In another embodiment of the present invention, a composition according to any of the dry powder composition embodiments described herein is provided wherein the composition is administered before, simultaneously or subsequently to the administration of an agent selected from clofazimine or a pharmaceutically acceptable salt or derivative thereof, cefoxitine, amikacin, clarithromycin, pyrazinamide, rifampin, moxiflxacin, levofloxacin and para-amino salicylate, and mixtures thereof. In a preferred embodiment of this embodiment, the composition is administered before, simultaneously or subsequently to the administration of an agent selected from clofazimine, or a pharmaceutically acceptable salt or derivative thereof, and amikacin, and mixtures thereof. In another preferred embodiment of this embodiment, the composition is administered before, simultaneously subsequently to administration of clofazimine. In another preferred embodiment of this embodiment, the composition is administered before, simultaneously subsequently to administration of amikacin.

In another embodiment of the present invention, a combination according to any of the pharmaceutical dry powder combinations herein described is provided wherein the pharmaceutical combination provided is used to administer before, simultaneously or subsequently to the administration of an agent selected from clofazimine or a pharmaceutically acceptable salt thereof, cefoxitine, amikacin, clarithromycin, pyrazinamide, rifampin, moxiflxacin, levofloxacin and para-amino salicylate, and mixtures thereof. In a preferred embodiment of this embodiment, the combination is used to administer before, simultaneously or subsequently to the administration of an agent selected from clofazimine, or a pharmaceutically acceptable salt or derivative thereof, and amikacin, and mixtures thereof. In another preferred embodiment of this embodiment, the combination is used to administer before, simultaneously or subsequently to the administration of an agent selected from clofazimine, or a pharmaceutically acceptable salt or derivative thereof, and amikacin, and mixtures thereof. In another preferred embodiment of this embodiment the combination is used to administer before, simultaneously or subsequently to the administration of clofazimine. In another preferred embodiment of this embodiment the combination is used to administer before, simultaneously or subsequently to the administration of amikacin.

In a further embodiment of the present invention a method of treatment or prophylaxis of a pulmonary infection caused by mycobacteria or other gram positive bacteria is provided, in a patient in need thereof, comprising administering by inhalation a composition of the present invention as described herein. In a preferred embodiment, the infection is caused by a species of the genus Mycobacterium selected from nontuberculous mycobacteria and Mycobacterium tuberculosis complex, and a combination thereof. In another preferred embodiment, a method of treatment or prophylaxis is provided wherein the nontuberculosis Mycobacterium is selected from Mycobacterium avium, Mycobacterium intracellulare, Mycobacterium abscessus, and Mycobacterium leprae, and a combination thereof. In another preferred embodiment, a method of treatment or prophylaxis is provided wherein the infection is an opportunistic infection, selected from MAC pulmonary disease and nontuberculosis infection, in a patient with cystic fibrosis, chronic obstructive pulmonary disease or acquired immune deficiency syndrome. In another preferred embodiment, a method of treatment or prophylaxis is provided wherein infection is an opportunistic nontuberculosis mycobacteria infection in a patient with cystic fibrosis.

In another embodiment of the present invention, a method of treatment or prophylaxis of a pulmonary infection caused by mycobacteria or other gram positive bacteria, in a patient in need thereof, is provided comprising administering by inhalation a composition according to the present invention as described herein, simultaneously, or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In a preferred embodiment, the agent is clofazimine or amikacin. In another preferred embodiment, the agent is clofazimine.

Suitable powders for use with dry powder inhalers may be comprised of micronized drug formed by processes known to the art such as jet milling, high pressure homogenization or spray drying. The drug may be delivered by itself or blended with pharmaceutical grade lactose (e.g. Lactohale®, DFE Pharma, Veghel, Netherlands). Blended formulations may include a tertiary component such as magnesium stearate as a release agent (Jetzer et al., “Investigations on the Mechanism of magnesium stearate to modify aerosol performance in dry powder inhaled formulations”, J. Pharm Sci, 107(4) 984-998, 2018).

Spray dried particles may be 100% drug or may contain one or more additional components to enhance the stability of the drug, or the dispersibility of the powder. In one embodiment of the present invention, the additional component is a sugar, for example, but not limited to, trehalose, sucrose, lactose or fructose. Combinations of sugars can also be employed. In another embodiment the spray dried particles of the invention can include one or more phospholipids. Specific examples of phospholipids include, but are not limited to phosphatidylcholines, dipalmitoyl phosphatidylcholine (DPPC), dipalmitoyl phosphatidylethanolamine (DPPE), distearoyl phosphatidylcholine (DSPC), dipalmitoyl phosphatidyl glycerol (DPPG), or any combination thereof. In another embodiment, the particles can contain an amino acid. Specific examples of suitable amino acids include, but are not limited to, leucine and isoleucine.

Optionally, the particles include, in addition to sugar or sugars, phospholipid or phospholipids, or amino acid or amino acids, a small amount of a strong electrolyte salt, such as, but not limited to, sodium chloride, sodium phosphate, sodium fluoride, sodium sulfate and calcium carbonate.

Suitable inhalers are described, for example, in U.S. Pat. Nos. 4,069,819; 4,995,385; and 5,997,848. Other examples include, but are not limited to, the SPINHALER® (Fisons), ROTAHALER® (Glaxo-Wellcome), FLOWCAPS® (Hovione), INHALATOR® (Boehringer lngelheim), AEROLIZER® (Novartis) and DISKHALER® (Glaxo-Wellcome), Plastiape RS-01® and others such as are known to those skilled in the art.

### Particle Size and Distribution

The therapeutic effect of aerosolized therapies is dependent upon the dose deposited and its distribution. Aerosol particle size is one of the important variables in defining the dose deposited and the distribution of drug aerosol in the lung.

Generally, inhaled aerosol particles are subject to deposition by one of two mechanisms: impaction, which usually predominates for larger aerosol particles, and sedimentation, which is prevalent for smaller aerosol particles. Impaction occurs when the momentum of an inhaled aerosol particle is large enough that the particle does not follow the air stream and encounters a physiological surface. In contrast, sedimentation occurs primarily in the lower lung when very small aerosol particles which have traveled with the inhaled air stream encounter physiological surfaces as a result of gravitational settling.

Pulmonary drug delivery may be accomplished by inhalation of an aerosol through the mouth and throat. Aerosol particles having an aerodynamic diameter of greater than about 5 μm generally do not reach the lung; instead, they tend to impact the back of the throat and are swallowed and possibly orally absorbed. Aerosol particles having diameters of about 3 μm to about 5 μm are small enough to reach the upper-to mid-pulmonary region (conducting airways), but are too large to reach the alveoli. Smaller aerosol particles, i.e. about 0.5 to about 3 μm, are capable of reaching the alveolar region. Aerosol particles having diameters smaller than about 0.5 μm tend to be exhaled during tidal breathing, but can also be deposited in the alveolar region by a breath hold.

Aerosols used in pulmonary drug delivery are made up of a wide range of aerosol particle sizes, so statistical descriptors are used. Aerosols used in pulmonary drug delivery are typically described by their mass median diameter (MMD), that is, half of the mass is contained in aerosol particles larger than the MMD, and half the mass is contained in aerosol particles smaller than the MMD. For particles with uniform density, the volume median diameter (VMD) can be used interchangeably with the MMD. Determinations of the VMD and MMD are made by laser diffraction. The width of the distribution is described by the geometric standard deviation (GSD). However, the deposition of an aerosol particle in the respiratory tract is more accurately described by the particle's aerodynamic diameter, thus, the mass median aerodynamic diameter is typically used. MMAD determinations are made by inertial impaction or time of flight measurements. For aqueous particles, VMD, MMD and MMAD should be the same. However, if humidity is not controlled as the aerosol transits the impactor, MMAD determinations will be smaller than MMD and VMD due to dehydration. For the purposes of this description, VMD, MMD and MMAD measurements are considered to be under controlled conditions such that descriptions of VMD, MMD and MMAD will be comparable.

Nonetheless, for the purpose of the description, the aerosol particle size of the aerosol particles will be given as MMAD as determined by measurement at room temperature with a Next Generation Impactor (NGI) in accordance with US Pharmacopeial Convention. In Process Revision <601> Aerosols, Nasal Sprays, Metered-Dose Inhalers, and Dry Powder Inhalers, Pharmacopeial Forum (2003), Volume Number 29, pages 1176-1210 also disclosed in Jolyon Mitchell, Mark Nagel “Particle Size Analysis of Aerosols from Medicinal Inhalers”, KONA Powder and Particle Journal (2004), Volume 22, pages 32-65.

In accordance with the present invention, the particle size of the aerosol is optimized to maximize the deposition of bedaquiline at the site of infection and to maximize tolerability. Aerosol particle size may be expressed in terms of the mass median aerodynamic diameter (MMAD). Large particles (e.g., MMAD>5 μm) tend to deposit in the extrathoracic and upper airways because they are too large to navigate bends in the airways. Intolerability (e.g., cough and bronchospasm) may occur from upper airway deposition of large particles.

Thus, in accordance with a preferred embodiment, the MMAD of the aerosol should be less than about 5 μm, preferably between about 1 and 5 μm, more preferably below 3 μm (<3 μm).

However, a guided breathing maneuver can be used to allow larger particles to pass through the extrathoracic and upper airways and deeper into the lungs than during tidal breathing which will increase the central and lower lung deposition of the aerosol. A guided breathing maneuver may be as slow as 100 ml/min. Thus, when used with a guided breathing maneuver, the preferred MMAD of the aerosol should be less than about 10 μm.

For a suspension delivered by nebulizer, an equally important factor (in addition to aerosol particle size) is the particle size and size distribution of the solid particles, in this case bedaquiline particle size and distribution. The size of a solid particle in a given aerosol particle must be smaller than the aerosol particle in which it is contained. A larger aerosol particle may contain one or more solid particles. Further, when dealing with dilute suspensions, a majority of aerosol particles may contain no solid particle. As a result, drug is preferentially contained in larger aerosol particles (see, for example, Finlay, et al., “Predicting regional lung dosages of a nebulized suspension: Pulmicort (budesonide)”, Particulate Science and Technology 15:243, 1997).

Because of this, it is desirable to have solid drug particles that are significantly smaller than the MMAD of the aerosol particles. For example, if MMAD of the aerosol particles is 3 μm, than a desired solid particle would be 1 μm, or smaller.

Another consideration, for example when using a vibrating mesh nebulizer, the formulation is pumped through orifices in a plate, which breaks up the suspension into droplets. It follows, then, that the solid particles must also be smaller than these orifices, in order to pass through.

Solid particle size in the suspension may be given by the mean size of the particles, and also by the distribution of the particles. D90 values indicate that 90% of the aerosol mass is contained in particles smaller than the D90.

### Nebulizer

For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Heathcare, Inc. and DeVilbiss Health Care, Inc. Vibrating mesh nebulizers rely upon either piezoelectric or mechanical pulses to respirable liquid droplets generate. Other examples of nebulizers for use with bedaquiline described herein are described in U.S. Pat. Nos. 4,268,460; 4,046,146; 4,649,911; 4,624,251; 5,164,740; 5,586,550; 5,758,637; 6,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,161,536; 6,557,549; 6,612,303; 6,962,151; 8,596,264, 8,720,435, 7,131,440, 8,739,777, 9,975,136; and 8,387,895, all of which are hereby incorporated by reference in their entirety. Commercial examples of nebulizers that can be used with the bedaquiline compositions described herein include Respirgard 11®, Aeroneb®, Aeroneb® Pro, and Aeroneb® Go produced by Aerogen; AERx® and AERx Essence™ produced by Aradigm; Porta-Neb®, Freeway Freedom™ Sidestream, Ventstream and I-neb produced by Respironics, Inc.; and PARI LCPlus®, PARI LC-Star®, and e-Flow7m produced by PARI, GmbH. Further non-limiting examples are disclosed in U.S. Pat. No. 6,196,219.

In accordance with the present invention, the pharmaceutical composition may be preferably aerosolized using a nebulizing device selected from an ultrasonic nebulizer, an electron spray nebulizer, a vibrating membrane nebulizer, a jet nebulizer or a mechanical soft mist inhaler.

It is preferred that the device controls the patient's inhalation flow rate either by an electrical or mechanical process.

In a further preferred embodiment, the aerosol production by the device is triggered by the patient's inhalation, such as with an AKITA device.

Preferred (commercially available) examples of the above nebulizers/devices to be used in accordance with the present invention are Vectura fox, Pari eFlow, Pari Trek S, Philips Innospire mini, Philips InnoSpire Go, Medspray device, Aeroneb Go, Aerogen Ultra, Respironics Aeroneb, Akita, Medspray Ecomyst and Respimat.

Use in Treatment and/or Prophylaxis

The pharmaceutical compositions and pharmaceutical combinations and systems according to the present invention are intended for the use in the treatment and/or prophylaxis of pulmonary infections caused by mycobacteria or other bedaquiline susceptible bacteria, such as Staphylococcus aureus (including methicillin-resistant and vancomycin intermediate-resistant strains), Streptococcus pneumoniae, and Enterococcus spp. The pharmaceutical compositions and pharmaceutical formulations of the present invention may also be used for the treatment and/or prophylaxis of pulmonary fungal infections.

### Dosing of Bedaquiline

In accordance with the present invention, the pharmaceutical composition is delivered by nebulization in about 1-5 ml, preferably 1-2 ml of the pharmaceutical composition of the invention.

Thus, the target fill dose is about 1-5 ml corresponding to 20-100 mg bedaquiline, based on a bedaquiline concentration in the pharmaceutical composition of about 20 mg/ml.

The daily lung dose (i.e. the dose deposited in the lung) of bedaquiline whether as a suspension from a nebulizer device, or as a dry powder from a dry powder inhaler, to be administered in accordance with the present invention is about 5-10 mg, which corresponds to a nominal dose of 15-30 mg (device dose) in the case of M. abscessus infections.

It is understood that the person of skill in the art will routinely adjust the lung dose of bedaquiline to be administered (and thus the fill/nominal dose/the volume to be nebulized) based on the MIC of bedaquiline for the respective bacteria strain well established in the art.

Depending on the dosing frequency, once or twice per day, the daily lung dose will be split accordingly.

In accordance with the present invention, bedaquiline is to be administered once or twice daily with a resulting total daily lung dose of about 5 to 10 mg.

It will be obvious to a person skilled in the art that the above amounts relate to bedaquiline free base, the dosage amounts for derivatives, and salts will have to be adjusted accordingly based on the MIC of the respective compound and strain.

### Mucolytic Agents/Biofilm Modifying Agents

In order to reduce sputum viscosity during aerosol treatment and to destroy existing biofilm, the treatment and/or prophylaxis according with the present invention can involve additional administration of mucolytic and/or biofilm destructing agents.

These agents can be prepared in fixed combination or be administered simultaneously or subsequently to the pharmaceutical composition/aerosol formulation comprising bedaquiline in accordance with the present invention.

Agents for dispersing/destruction of the biofilm, mucolytic and/or mucoactive agents and/or agents that reduce biofilm formation to be used in accordance with the present invention are selected from nebulized 4-7% hypertonic saline, metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides.

Furthermore, also other pharmaceutically active agents may be used in combination with the pharmaceutical compositions/aerosol formulations in accordance with the present invention. Such active agents may be selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

These agents can be prepared in fixed combination or be administered prior to, simultaneously or subsequently to the pharmaceutical composition/aerosol formulation comprising bedaquiline in accordance with the present invention.

### EXAMPLES

The following examples serve to more fully describe the manner of using the above-described invention, as well as to set forth the best modes contemplated for carrying out various aspects of the invention. The Examples according to the invention are those falling within the scope of the claims herein.

### Experimental

The exemplary compositions and formulations below have been prepared in accordance with the processes described herein.

### Example 1

Preparation of suspension Composition 1.

A suspension was prepared having the following composition:

500 mg bedaquiline

2.5 ml Polysorbate 80 (NOF Corporation Hx2)

450 mg sodium chloride

47.5 ml water

At this point, the median size of the bedaquiline particles was 14.13 μm, with a D90 of 103.48 μm, as determined using Horiba LA950.

Homogenization was initiated with a Polytron® Immersion Dispenser PT 2500 E (Kinematica, Luzern, Switzerland), with 2×5 minute treatments at 10,000 rpm. The resulting suspension was then homogenized using Branson Digital Sonifier 250D, Model 102C with 7×3 minute treatments at 70%, while on ice.

The resulting suspension had a pH of 6.91, and an osmality of 341 mOsmol/kg, as determined with a SEMI MICRO Osmometer K-74OO (Knauer).

The median size of the bedaquiline particles was determined to be 3.96 μm, with a D10 of 2.29 μm and a D90 of 6.18 μm.

Determination of Minimum Inhibitory Concentration (MIC)

Drug susceptibility testing was performed as advised by the Clinical and Laboratory Standards institute. This was performed by broth microdilution in cation-adjusted Mueller-Hinton broth for Mycobacterium abscessus and by the broth macrodilution method, using the BacTec460 for Mycobacterium avium. MICs were determined by testing susceptibility to concentrations of the Composition of Example 1, between 0.05 μg/ml and 8 μg/ml. Results are shown in Table 2. M. avium B16079517 and M. abscessus B15012958 are clinical isolates. M. avium ATCC700898 and M. abcessus CIP104536 are commercial strains.

| Species | MIC μg/ml) |
|---|---|
| M. avium (ATCC 700898) | 0.03 |
| M. avium (B16079517) | 0.03 |
| M. abscessus (CIP104536) | 0.125 |
| M. abscessus(B15012958) | 0.5 |

These results indicate that the Composition of Example 1 shows significant inhibitory activity against these mycobacteria.

### Preparation of Further Compositions of Bedaquiline

A suspension of bedaquiline was prepared having the following composition:

40 mg bedaquiline

2.0 ml Polysorbate 80 (NOF Corporation Hx2)

360 mg sodium chloride

398 ml water

At this point, the mean size of the bedaquiline particles was 9.30 μm, with a D90 of 10.97 μm, as determined using a Horiba LA950.

This suspension was added to a M-110EH-30 Microfluidizer® Processor using an

H30Z Interaction Chamber. This suspension was recirculated for 5 minutes at 4,400 psi. At this point, the mean size of the bedaquiline particles was 2.80 μm, with a D90 of 4.41 μm.

A G1OZ Interaction Chamber was installed, and the above suspension resulting from the H30Z chamber was recirculated at 25,000 rpm, collecting samples at 10, 20 and 35 minutes, with the following results: (1) after 10 minutes the mean size of the bedaquiline particles was 0.95 μm, with a D90 of 2.08 μm; (2) after 20 minutes the mean size of the bedaquiline particles was 0.46 μm, with a D90 of 1.16 μm; and (3) after 35 minutes the mean size of the bedaquiline particles was 0.30 μm, with a D90 of 0.79 μm. The pH of this 35 minute sample was 6.431, with an osmolality of 297 mOsmol/kg.

Stability of the 35 minute suspension described above was determined after standing for 17 days. Measurements done in triplicate indicated a mean size of the bedaquiline particles of 0.37 μm, with a D90 of 0.96 μm.

Cell Viability

Two different cell types were used to assess pulmonary epithelial cell viability in the presence of bedaquiline suspensions. The cell lines were A549 (DSMZ; ACC107) and Calu-3 (LGC standards, ATCC-HTB-55).

A549 cells were cultivated in Roswell Park Memorial Institute Medium (RPMI 1640) plus 10% Fetal Calf Serum (FCS), 1% Penicillin/streptomycin (10,000 units/ml Penicillin; 10,000 units/ml Streptomycin) (Pen/Strep), and the Calu-3 cells were cultivated in Minimum Essential Medium (Gibco by life technologies) (MEM) plus 10% FCS, 1% Non-essential amino acids (a supplement for MEM), 1% sodium pyruvate, and 1% Pen/Strep. For routine cell culture, the A549 and Calu-3 cells were passaged once a week at a confluence of 80-90%, as follows: The cells were cultivated in 175 cm2 cell culture flasks and were washed once with 10 ml 1 time Dulbecco's phosphate-buffered saline (DPBS). After incubation with 0.05% Trypsin-EDTA for 5 minutes (A549) or 15 minutes with additional cell scraping (Calu-3) at 37° C., the cells were centrifuted for 5 minutes at 300 g. The cell pellet was re-suspended in the respective cell culture medium. The cells were counted a Luna cell counter by using 10 μI of stained cell suspension (18 μI cell suspension plus 2 μI Acridine-Orange, Live-Dead staining). For routine culture, 230,000 cells per flask (A549) or, respectively, 1,300,000 cells per flask (Calu-3) were seeded in a new T175 cm² flask cultivated at 37° C. with 5% CO₂ atmosphere.

MTT is the tetrazolium dye 3-(4,5-dimethylthiazol-2-yl)-2,5-diphenyltetrazolium bromide which is converted by mitochondrial reductase to its insoluble formazan. In living cells, the insoluble formazan can be dissolved by adding the detergent dimethylsulfoxide for 15 minutes. The absorption of the respective dye is measured by a plate-reader at 590 nm. For the calculation of the viability after the incubation of the test compounds a positive and a negative control is included. Hank's balanced salt solution (HBSS) is used as a negative control and the resulting absorbance value is set to 100% viability. The positive control (1% Triton-X-100) is used to set 0% viability. Accordingly, the IC50 value of the test formulation can be determined by measuring a dose-response curve in log-scale.

Test samples were prepared for suspensions of bedaquiline, and for a vehicle containing no bedaquiline as follows:

A formulation of bedaquiline was prepared containing bedaquiline at 1 mg/ml, polysorbate 80 at 0.5%, sodium chloride at 0.9% in distilled water. The formulation for vehicle containing no bedaquiline was prepared containing 0.5% polysorbate 80 in distilled water, or containing 0.5% polysorbate 80, and 0.9% sodium chloride in water.

These formulations (designated as 100%) were diluted with HBSS to give the following formulations for testing:

Concentrations of Vehicle Solutions

| Concentration (%) | Vehicle Solution (μl) | HBSS (μl) |
|---|---|---|
| 100 | 2000 | 0 |
| 95 | 1900 | 100 |
| 90 | 1800 | 200 |
| 85 | 1700 | 300 |
| 80 | 1600 | 400 |
| 70 | 1400 | 600 |
| 60 | 1200 | 800 |
| 50 | 1000 | 1000 |

Concentrations of Bedaquiline Containing Suspensions

| Concentration | Bedaquiline (μl) | | Bedaquiline |
|---|---|---|---|
| (%) | suspension | HBSS (μl) | (mg/ml) |
| 100 | 2000 | 0 | 1 |
| 90 | 1800 | 200 | 0.9 |
| 80 | 1600 | 400 | 0.8 |
| 70 | 1400 | 600 | 0.7 |
| 60 | 1200 | 800 | 0.6 |
| 50 | 1000 | 1000 | 0.5 |
| 40 | 800 | 1200 | 0.4 |
| 30 | 600 | 1400 | 0.3 |
| 20 | 400 | 1600 | 0.2 |
| 10 | 200 | 1800 | 0.1 |
| 1 | 20 | 1980 | 0.01 |

Cells were treated with the test formulations for 4 hours at 37° C. The viability of the cells after exposure was used to set up a dose-response curve in logarithmic scale. A sigmoidal fit enables the IC20, IC50, and IC80 calculations of the test substances. This is done in the statistical program Origin®Pro 2019.

### Results

A549

| | Bedaquline | Vehicle |
|---|---|---|
| IC VALUE | Suspensions | Solutions |
| IC2O | 90-95% | 90-95% |
| IC5O | 85-90% | 85-90% |
| IC8O | 80-85% | 85-90% |

Calu-3

| | Bedaquline | Vehicle |
|---|---|---|
| IC VALUE | Suspensions | Solutions |
| IC2O | 95-100% | 95-100% |
| IC5O | 80-95% | 95-100% |
| IC8O | 60-70% | 95-100% |

These data indicate that bedaquiline has a minimal cytotoxic effect on cell viability as compared with the particular vehicle tested.

1. A pharmaceutical composition comprising:
a therapeutically effective dose of bedaquiline or a pharmaceutically acceptable derivative or salt thereof;
a nonionic surfactant with an Hydrophilic-Lipophilic Balance value of greater than 10; and
an aqueous liquid carrier selected from water, isotonic saline, buffered saline and aqueous electrolyte solutions
wherein the bedaquiline or the pharmaceutically acceptable derivative or salt thereof is provided in the form of particles in a suspension, and
wherein the bedaquiline particles, or the particles of the pharmaceutically acceptable salt of bedaquiline, have a median size of less than 5 μm and a D90 of less than 6.5 μm.

2. A pharmaceutical composition according to claim 1 wherein the particles of bedaquiline, or the pharmaceutically acceptable salt thereof, have a median size of less than 2 μm and a D90 of less than 3 μm.

3-4. (canceled)

5. A pharmaceutical composition according to claim 1, wherein the nonionic surfactant is selected from polysorbate 20, polysorbate 60, polysorbate 80, stearyl alcohol, a polyethylene glycol derivative of hydrogenated castor oil with an Hydrophilic-Lipophilic Balance value of 14 to 16, a polyethylene 10 glycol derivative of hydrogenated castor oil with an Hydrophilic-Lipophilic Balance value of 15 to 17, sorbitan monolaurate, sorbitan monopalmitate, sorbitan monostearate, polyoxyethylene (20) oleyl ether, polyoxyethylene (20) cetyl ether, polyoxyethylene (10) cetyl ether, polyoxyethylene (10) oleyl ether, polyoxyethylene (100) stearyl ether, polyoxyethylene (10) stearyl ether, polyoxyethylene (20) stearyl ether, polyoxyethylene (4) lauryl ether, polyoxyethylene (20) cetyl ether, polyoxyethylene (2) cetyl ether, caprylocaproyl polyoxyl-8 glyceride, polyethylene glycol (20) monostearate, polyethylene glycol (40) stearate, polyethylene glycol (100) stearate, polyethylene glycol (8) stearate, and polyoxyl 40 stearate, and mixtures thereof.

6. A pharmaceutical composition according to claim 1, wherein the non-ionic surfactant is polysorbate 80, and wherein the aqueous liquid carrier is distilled water, hypertonic saline or isotonic saline.

7. A pharmaceutical composition according to claim 6, wherein the hypertonic saline is from 1% to 7% (w/v) sodium chloride.

8. A pharmaceutical composition according to claim 6, wherein the non-ionic surfactant is ultrapure polysorbate 80, and wherein the aqueous liquid carrier is isotonic saline.

9. A pharmaceutical composition according to claim 1 wherein the osmolality of the composition is in the range of 200-700 mOsm/kg.

10. A pharmaceutical composition according to claim 1 and further comprising about 500 mg bedaquiline, about 2.5 ml Polysorbate 80, about 450 mg sodium chloride, and about 47.5 ml water wherein the osmolality of the composition is in the range of 300-400 mOsm/kg, and the median size of the bedaquiline particles was 14.13 μm, with a D90 of 103.48 μm, as determined using Horiba LA950.

11. A pharmaceutical composition according to claim 1, wherein the concentration of nonionic surfactant is in the range of 0.001% to 5% (v/v) of the total composition and the amount of bedaquiline is in the range of 0.1% to 20% (w/v) of the total composition.

12. A pharmaceutical composition according to claim 1, prepared by a process comprising the following steps:
1. homogenization of a suspension of bedaquiline, the nonionic surfactant and water to obtain a suspension comprising bedaquiline of an appropriate particle size,
2. adjusting the pH of the suspension resulting from (1) to a pH of between pH 5.5 and pH 7.5,
3. adjusting the sodium chloride concentration to an appropriate concentration and
4. adjusting the osmolality to an appropriate level.

13-15. (canceled)

16. A pharmaceutical composition prepared by the process according to claim 12, wherein the pH is adjusted to 6.5, and the sodium chloride concentration is adjusted to 154 mM sodium chloride.

17-19. (canceled)

20. A pharmaceutical composition prepared by the process according to claim 1, wherein the homogenization in step (1) is carried out by high pressure homogenization, high shear homogenization, wet milling, ultrasonic homogenization, or a combination of such processes.

21. A pharmaceutical composition prepared by the process according to claim 12, wherein the homogenization of bedaquiline is carried out in multiple steps of homogenization.

22. (canceled)

23. A pharmaceutical composition prepared by the process according according to claim 12, wherein the appropriate particle size of the bedaquiline are particles having a mean size of less than 5 μm and D90 of less than 6.5 μm.

24. A pharmaceutical composition prepared by the process according to claim 12, wherein the appropriate particle size of the bedaquiline are particles having a mean size of less than 2 μm and D90 of less than 3 μm.

25. A pharmaceutical combination in the form of an aerosol for inhalation, prepared by aerosolization of the composition according to claim 1 by a nebulizing device selected from an ultrasonic nebulizer, an electron spray nebulizer, a vibrating membrane nebulizer, a jet nebulizer and a mechanical soft mist inhaler, and wherein the aerosol particles produced by the nebulizing device have a mass median aerodynamic diameter of 1 to 5 μm.

26-38. (canceled)

39. A system for use in providing antibiotic activity when treating or providing prophylaxis against a pulmonary infection caused by mycobacteria or other gram-positive bacteria, wherein the system comprises:
a nebulized pharmaceutical formulation comprising:
a therapeutically effective dose of bedaquiline;
a nonionic surfactant with an Hydrophilic-Lipophilic Balance value of greater than 10; and
an aqueous liquid carrier selected from water, isotonic saline, buffered saline and aqueous electrolyte solutions and
a nebulizer, wherein the bedaquiline is present in the form of a suspension, and wherein the aerosol particles produced by the system have a mass median aerodynamic diameter of 1 to 5 μm.

40-78. (canceled)

79. A pharmaceutical composition for dry powder inhalation comprising bedaquiline, or a pharmaceutically acceptable salt or derivative thereof, of an appropriate particle size, and a physiologically acceptable pharmacologically inert excipient, or a mixture of physiologically acceptable pharmacologically inert excipients of appropriate particle size or sizes.

80-104. (canceled)

105. A system for use in providing antibiotic activity when treating or providing prophylaxis against a pulmonary infection caused by mycobacteria or other gram-positive bacteria, wherein the system comprises:
a dry powder pharmaceutical formulation comprising
a) a therapeutically effective dose of bedaquiline,
b) one or more excipients selected from sugars, amino acids, and phospholipids, and combinations thereof,
a container for the formulation selected from a capsule or blister package, and
a dry powder inhaler,
wherein the bedaquiline is present in the form of a dry powder, and wherein the bedaquiline containing particles have a mass median diameter of 1 to 5 μm.

106-112. (canceled)

113. A method of treatment or prophylaxis of a pulmonary infection caused by mycobacteria or other gram positive bacteria, in a patient in need thereof, comprising administering by inhalation a composition according to claim 79, wherein the composition comprises less than 100 mg of bedaquiline.

114. A method of treatment or prophylaxis according to claim 113, wherein the infection is caused by a species of the genus Mycobacterium selected from nontuberculous mycobacteria and Mycobacterium tuberculosis complex, and a combination thereof.

115. A method of treatment or prophylaxis according to claim 114, wherein the nontuberculosis Mycobacterium is selected from Mycobacterium avium, Mycobacterium intracellulare, Mycobacterium abscessus, and Mycobacterium leprae, and a combination thereof.

116. A method of treatment or prophylaxis according to claim 113, wherein the infection is an opportunistic infection, selected from MAC pulmonary disease and nontuberculosis infection, in a patient with cystic fibrosis, chronic obstructive pulmonary disease or acquired immune deficiency syndrome.

117. A method of treatment or prophylaxis according to claim 116, wherein infection is an opportunistic nontuberculosis mycobacteria infection in a patient with cystic fibrosis.

118. A method of treatment or prophylaxis of a pulmonary infection caused by mycobacteria or other gram positive bacteria, in a patient in need thereof, comprising administering by inhalation a composition according to claim 79, before, simultaneously, or subsequently to the administration of an agent selected from clofazimine or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

119. A method of treatment or prophylaxis according to claim 118, wherein the agent is clofazimine or amikacin.

120. A method of treatment or prophylaxis according to claim 119, wherein the agent is clofazimine.
